(19) United States
(12) Patent Application Publication
(10) Pub. No.: US 2022/0223239 A1
(43) Pub. Date: Jul. 14, 2022

(54) DETERMINATION DEVICE, DETERMINATION METHOD, AND DETERMINATION PROGRAM

(71) Applicant: SAITAMA MEDICAL UNIVERSITY, Saitama (JP)

(72) Inventor: Koshiro NISHIMOTO, Saitama (JP)

(21) Appl. No.: 17/609,865

(22) PCT Filed: May 7, 2020
(86) PCT No.: PCT/JP2020/018537

§ 371 Date: Nov. 9, 2021

(30) Foreign Application Priority Data
May 9, 2019 (JP) 2019-089266

(51) Int. Cl. G16H 10/40; A61B 6/03; G06T 7/00

(52) U.S. Cl. CPC G16H 10/40; A61B 6/03; G06T 2207/20076; G06T 2207/30096; G06T 2207/10081; G06T 7/0012

(57) ABSTRACT

A determination device includes a factor information acquisition unit configured to acquire information on one or more predetermined factors for a subject with a primary aldosteronism, a tumor diameter acquisition unit configured to acquire a tumor diameter, the tumor diameter being a size of a largest tumor among tumors included in an image of an adrenal gland of the subject obtained through computed tomography, and a calculation unit configured to calculate a probability of an aldosterone-producing adenoma of the adrenal gland of the subject being smaller than a predetermined size on the basis of the information of one or more predetermined factors acquired by the factor information acquisition unit, and determine that adrenalectomy is indicated for the subject on the basis of the tumor diameter and the probability.

### TECHNICAL FIELD

The present invention relates to a determination device, a determination method, and a determination program.

### BACKGROUND ART

Primary aldosteronism (PA) is a disease in which excess aldosterone is produced from an adrenal cortex, resulting in severe hypertension through retention of salt or water. It is known that, in addition to hypertension, aldosterone acts directly on blood vessels to promote fibrosis or remodeling of the blood vessels, and thus, causes cerebrocardiovascular complications, especially frequent strokes. Such PA is a frequent secondary hypertensive disease that accounts for about 10% of hypertensive patients.

### CITATION LIST

### Patent Literature

[PTL 1] Japanese Translation of PCT Application No. 2017-500538
[PTL 2] Japanese Translation of PCT Application No. 2013-534911
[PTL 3] WO 2014/054675
[PTL 4] WO 2017/175494
[PTL 5] WO 2012/157130

### SUMMARY OF INVENTION

### Technical Problem

PA is mainly classified into unilateral aldosterone-producing adenoma (APA) and bilateral idiopathic hyperaldosteronism (IHA). The former is cured by adrenal gland resection in which one of adrenal glands is resected, whereas the latter requires lifelong cure using a mineralocorticoid receptor (aldosterone receptor) inhibitor. As a method of determining whether PA is unilateral APA or bilateral IHA, detection of an adrenal tumor using computed tomography (CT) or adrenal venous sampling (AVS) is performed. Although the detection of an adrenal tumor using CT is non-invasive, adrenal incidentaloma tumors (tumors without hormone production) are frequently erroneously detected and thus, a specificity thereof is about 70%. On the other hand, AVS is a method in which a catheter is inserted into the left and right narrow adrenal veins under X-ray fluoroscopy, blood is collected from the adrenal veins, left and right aldosterone-cortisol ratios are compared with each other, and unilateral APA is diagnosed when a left and right ratio is equal to or larger than 4 (this value depends on guidelines). AVS is a testing method that requires advanced technology, and a test cost of AVS is high. Further, AVS has a problem that a burden on a patient's body is large. Therefore, it is preferable to detect unilateral APA without performing AVS as far as possible.

An object of the present invention is to provide a technology for determining that adrenalectomy is indicated from a patient with primary aldosteronism.

### Solution to Problem

In order to solve the above problem, the following means is adopted.

That is, a first aspect is a determination device including: a factor information acquisition unit configured to acquire information on one or more predetermined factors for a subject with primary aldosteronism; a tumor diameter acquisition unit configured to acquire a tumor diameter, the tumor diameter being a size of a largest tumor among tumors included in an image of an adrenal gland of the subject obtained through computed tomography; and a calculation unit configured to calculate a probability of an aldosterone-producing adenoma of the adrenal gland of the subject being smaller than a predetermined size on the basis of the information of the one or more predetermined factors acquired by the factor information acquisition unit, and determine whether adrenal venous sampling is required for the subject on the basis of the tumor diameter and the probability.

### Advantageous Effects of Invention

According to the present invention, it is possible to provide a technology for determining that adrenalectomy is indicated for a patient with primary aldosteronism.

### BRIEF DESCRIPTION OF DRAWINGS

FIG. 1 is a table illustrating an example of a comparison between postoperative PA cure rates in a case in which threshold values of L-PAL and S-PAL were changed.

FIG. 2 is a diagram illustrating a configuration example of a determination device according to an embodiment.

FIG. 3 is a diagram illustrating a hardware configuration example of an information processing device.

FIG. 4 is a diagram illustrating an example of an operation flow for calculation of a probability p of a tumor of an adrenal gland of a subject being S-PAL in the determination device.

FIG. 5 is a diagram illustrating actual cases.

### DESCRIPTION OF EMBODIMENTS

Hereinafter, embodiments will be described with reference to the drawings. A configuration of the embodiment is an example, and a configuration of the invention is not limited to a specific configuration of the disclosed embodiment. A specific configuration according to the embodiment may be appropriately adopted in implementing the invention.

### Embodiment

### (Overview)

An immunostaining method (a staining method for distinguishing aldosterone synthase [CYP11B2] from cortisol synthase [CYP11B1] and detecting the aldosterone synthase) for specifically detecting aldosterone-producing cells (aldosterone-producing adenoma) has been developed and pathological definitive diagnosis for APA has been performed. Small and frequent aldosterone-producing cell clusters (APCCs) or possible APCC-to-APA transitional lesions (pAATLs) have been detected in non-PA adrenal glands or PA adrenal glands with unclear tumors. When a tumor is found in an adrenal gland in a CT image in a PA patient, but the tumor is an accidental tumor, most lesions of PA have been found to be frequent APCCs or pAATLs. Therefore, when a method of preoperatively predicting APA and APCCs or pAATLs is established, it is possible to predict whether the PA case with a tumor is APA or an accidental tumor and APCCs/pAATLs in the CT image.

Here, largest lesions among CYP11B2 positive lesions due to CYP11B2 staining after adrenalectomy are classified into a large PA-lesion (L-PAL) that is equal to or larger than a threshold value and a small PA-lesion (S-PAL) that is smaller than a threshold value. Here, the threshold values of L-PAL and S-PAL are set to 5 mm. As a result, it has been found that in L-PAL, a postoperative PA cure rate is significantly (P<0.05) high, and in S-PAL, postoperative PA persistence rates are all high. This is presumed to be because S-PAL is bilateral from the fact that L-PAL is often unilateral and S-PAL is often bilateral.

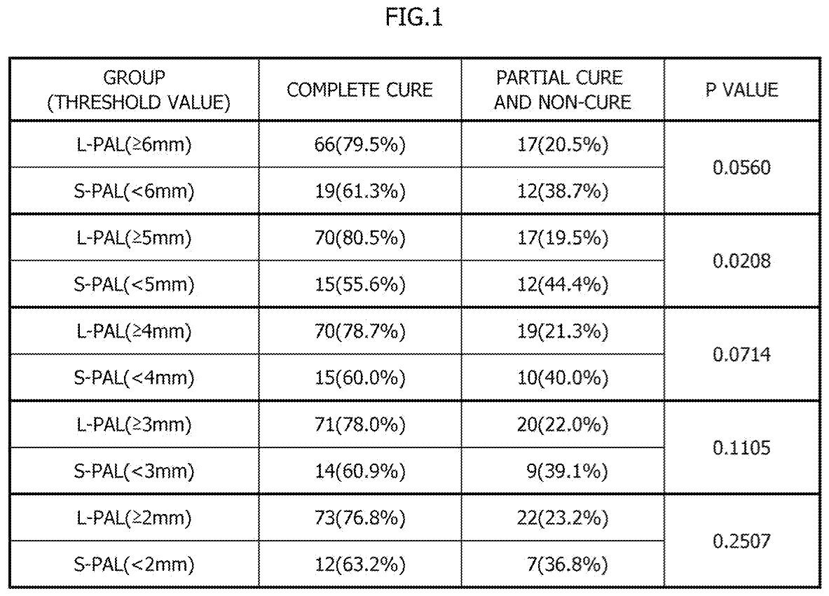

FIG. 1 is a table illustrating an example of a comparison between postoperative PA cure rates in a case in which the threshold values of L-PAL and S-PAL were changed. In the table of FIG. 1, the number of cases and percentages of postoperative complete cure and postoperative partial cure or non-cure when the threshold values of L-PAL and S-PAL were 6 mm, 5 mm, 4 mm, 3 mm, and 2 mm are compared between L-PAL and S-PAL. The proportions are proportions of a total number of L-PAL or S-PAL cases (complete cure, partial cure, and non-cure). For example, when the threshold value was set to 6 mm, 66 cases (79.5%) among L-PAL cases were completely cured, and 17 cases (20.5%) were partially cured or not cured. Further, when the threshold value was set to 6 mm, 19 cases (61.3%) among S-PAL cases were completely cured, and 12 cases (38.7%) were partially cured or not cured. Further, for each threshold value, a P value indicating a significance of a difference between L-PAL and S-PAL is calculated. In the example of the table of FIG. 1, when the threshold value was set to 5 mm, a rate of complete cure of L-PAL is highest and the P value is lowest. Therefore, it can be seen that it is preferable for the threshold value of L-PAL and S-PAL to be set to 5 mm. Here, patients with a postoperative aldosteron-to-renin ratio (ARR) smaller than 200 and a serum potassium value equal to or larger than 3.5 mEq/L are considered to have been completely cured. Further, patients with a postoperative ARR equal to or larger than 200, a preoperative ARR smaller than 50%, and a serum potassium value equal to or larger than 3.5 mEq/L are considered to have been partially cured. Further, the remaining patients are considered not to have been cured. Threshold values for L-PAL and S-PAL may be other values at which a postoperative PA cure rate of patients with L-PAL is significantly higher than a postoperative PA cure rate of patients with S-PAL. The threshold value can be determined on the basis of actual cases.

Independent preoperative factors for predicting S-PAL (calculating a probability of being S-PAL) are revealed by a correspondence between preoperative factors and whether or not a removed adrenal gland is S-PAL (which is a PA lesion having a size equal to or smaller than a threshold value (5 mm)), univariate analysis, and multivariate logistic regression analysis. Examples of candidates for independent preoperative factors for predicting S-PAL include age, gender, body mass index (BMI), blood pressure, a serum potassium value, a plasma aldosterone concentration, and a plasma renin activity. The serum potassium value, the plasma aldosterone concentration, and the plasma renin activity are quantities that can be measured by a blood test. Statistical analysis has revealed that independent preoperative factors for predicting S-PAL are the serum potassium value and the plasma aldosterone concentration. From these factors, a regression formula for calculating the probability of being S-PAL is obtained. Statistical analysis schemes other than univariate analysis and multivariate logistic regression analysis may be used to calculate a formula for calculating the probability of being S-PAL. A low probability of being S-PAL indicates a high probability of being L-PAL. The probability of being S-PAL is synonymous with a probability of not being L-PAL.

A regression formula for calculating the probability p of being S-PAL is expressed as follows, for example, using the serum potassium value and the plasma aldosterone concentration, which are independent preoperative factors for predicting S-PAL.

$$p = \frac{1}{1 + exp [ - ( A \cdot PAC + B \cdot K + C ) ]} \qquad [ Math . 1 ]$$

Here, PAC is a preoperative plasma aldosterone concentration (pg/mL). K is a preoperative serum potassium value (mEq/L). Further, the variables A, B, and C are coefficients obtained by univariate analysis and multivariate logistic regression analysis. From actual cases, for example, A=−0.00374, B=1.730, and C=−5.902 can be obtained. From this formula, it is possible to calculate the probability p of a tumor of the adrenal gland being S-PAL from the serum potassium value and the plasma aldosterone concentration measured preoperatively. The variables A, B, and C are not limited to the values listed here and can be determined on the basis of actual cases. Further, the independent preoperative factors for predicting S-PAL are not limited to the serum potassium values and the plasma aldosterone concentration. A preoperative factor may be the serum potassium value or the plasma aldosterone concentration. Alternatively, the preoperative factors may be one or more preoperative factors determined on the basis of the statistical analysis as described above (for example, a preoperative factor having a P value smaller than 0.001). In these cases, the regression formula for calculating the probability p of being S-PAL is calculated on the basis of a well-known statistical analysis scheme according to the determined preoperative factors.

Further, since the detection sensitivity and specificity for a pathological tumor by CT remains unclear, a study on a threshold value (2 mm to 10 mm) of a diameter of the adrenal tumor on a CT image (tumor in CT: T-CT) was conducted by statistical analysis based on comparison with a size of a PA-lesion in the removed adrenal gland. Specifically, when an area under a receiver operating characteristic (ROC) curve (AUC) was calculated and a predicted value at each threshold value was estimated, the AUC was largest at a threshold value of 3 to 5 mm (AUC=0.691). As a result, it was found that a threshold value of T-CT is 5 mm, which is one of the best threshold values for prediction of a threshold value of 5 mm in pathology. Here, the tumor diameter (T-CT) on the CT image is a size of the largest tumor in the CT image.

### Configuration Example

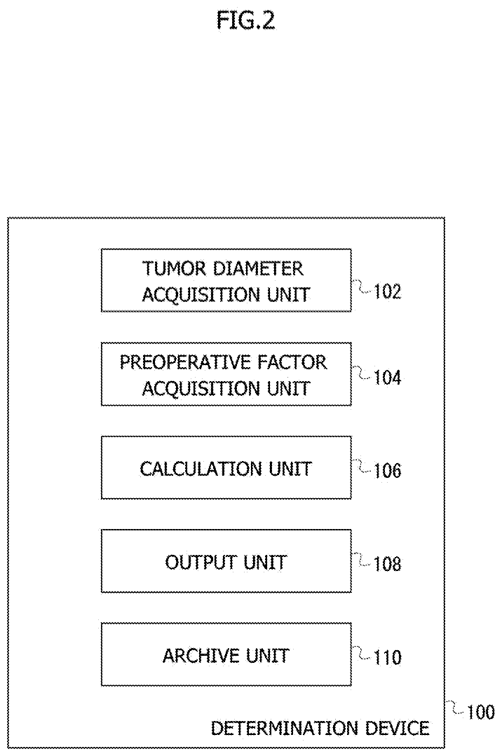

FIG. 2 is a diagram illustrating a configuration example of a determination device of the present embodiment. The determination device 100 includes a tumor diameter acquisition unit 102, a preoperative factor acquisition unit 104, a calculation unit 106, an output unit 108, and an archive unit 110. The determination device 100 is a device for calculating a probability p of a tumor of the adrenal gland of a human body (subject) being S-PAL.

The tumor diameter acquisition unit 102 acquires a size (tumor diameter) of the tumor of the adrenal gland in the CT image of the adrenal gland of the human body (subject) taken by a computed tomography device. The tumor diameter acquisition unit 102 acquires a size (tumor diameter) of a largest tumor among adrenal tumors on the CT image as a tumor diameter (tumor in CT; T-CT) of the adrenal gland on the CT image. The tumor diameter acquisition unit 102 acquires the tumor diameter of the adrenal gland of the subject in the CT image from the computed tomography device or the like connected to the determination device 100. The tumor diameter acquisition unit 102 may acquire a CT image in which a part including the adrenal gland of the subject has been imaged to calculate the tumor diameter of the largest tumor among the adrenal tumors in the CT image through known image processing and acquire the tumor diameter of the adrenal gland in the CT image. Further, the tumor diameter acquisition unit 102 may acquire the tumor diameter by causing a user (a doctor or the like) to input the tumor diameter of the adrenal gland in the CT image of the subject (the size of the largest tumor among the tumors of the adrenal gland in the CT image) using, for example, an input means such as a keyboard or a touch panel. The acquired tumor diameter is stored in the archive unit 110.

The preoperative factor acquisition unit 104 acquires the serum potassium value of the subject and the plasma aldosterone concentration of the subject, which are the independent preoperative factors for predicting S-PAL. The preoperative factor acquisition unit 104 acquires the serum potassium value of the subject and the plasma aldosterone concentration of the subject from a blood testing device or the like connected to the determination device 100. Further, the preoperative factor acquisition unit 104 may acquire the preoperative factors by causing a user (a doctor or the like) to input the serum potassium value of the subject and the plasma aldosterone concentration of the subject using, for example, an input means such as a keyboard or a touch panel. The acquired preoperative factors are stored in the archive unit 110. Here, the preoperative factors are the serum potassium value of the subject and the plasma aldosterone concentration of the subject, but other factors relevant to whether or not the tumor of the adrenal gland is S-PAL on the basis of statistical analysis and the like may be included.

The calculation unit 106 calculates the probability p of the tumor of the adrenal gland of the subject being S-PAL on the basis of the size of the tumor acquired by the tumor diameter acquisition unit 102 of the subject and the preoperative factors acquired by the preoperative factor acquisition unit 104. The calculated probability p is stored in the archive unit 110. The calculation unit 106 determines whether or not the tumor diameter of the adrenal gland of the subject in the CT image acquired by the tumor diameter acquisition unit 102 is equal to or larger than a predetermined value. Further, the calculation unit 106 determines whether or not the calculated probability p is equal to or larger than a predetermined value. Further, the calculation unit 106 determines whether or not AVS is required for the subject on the basis of the probability p and the tumor diameter T-CT of the adrenal gland in the CT image.

The output unit 108 displays the probability p of the tumor of the adrenal gland of the subject calculated by the calculation unit 106 being S-PAL on a display means such as a display.

The archive unit 110 stores the tumor diameter (T-CT) acquired by the tumor diameter acquisition unit 102, the preoperative factors such as the serum potassium value of the subject and the plasma aldosterone concentration of the subject acquired by the preoperative factor acquisition unit 104, the probability p calculated by the calculation unit 106, and the like. The archive unit 110 stores values, data, tables, calculation formulas, and the like used in the determination device 100.

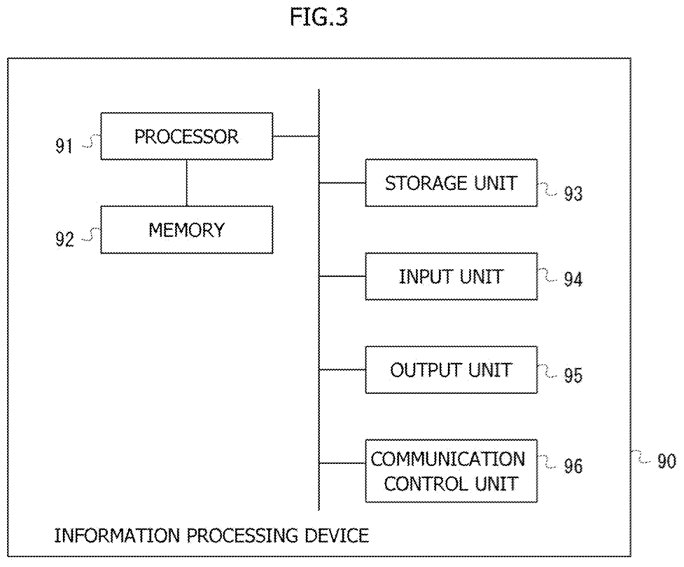

FIG. 3 is a diagram illustrating a hardware configuration example of the information processing device. An information processing device 90 illustrated in FIG. 3 has a configuration of a general computer. The determination device 100 is realized by using the information processing device 90 as illustrated in FIG. 3. The information processing device 90 of FIG. 3 includes a processor 91, a memory 92, a storage unit 93, an input unit 94, an output unit 95, and a communication control unit 96. These are connected to each other by a bus. The memory 92 and the storage unit 93 are computer-readable recording media. A hardware configuration of the information processing device is not limited to the example illustrated in FIG. 3, and components may be omitted, replaced, or added appropriately.

The information processing device 90 can realize a function satisfying a predetermined purpose by the processor 91 loading the program stored in the recording medium into a work area of the memory 92 and executing the program, and each component or the like being controlled through the execution of the program.

The processor 91 is, for example, a central processing unit (CPU) or a digital signal processor (DSP).

The memory 92 includes, for example, a random access memory (RAM) or a read only memory (ROM). The memory 92 is also called a main storage device.

The storage unit 93 is, for example, an erasable programmable ROM (EPROM) or a hard disk drive (HDD). Further, the storage unit 93 can include a removable medium, that is, a portable recording medium. The removable media is, for example, a universal serial bus (USB) memory or a disc recording medium such as a compact disc (CD) or a digital versatile disc (DVD). The storage unit 93 is also called a secondary storage device.

The storage unit 93 stores various programs, various pieces of data, and various tables used in the information processing device 90 in a recording medium so that various programs, various pieces of data, and various tables can be freely read or written. An operating system (OS), various programs, various tables, and the like are stored in the storage unit 93. Information stored in the storage unit 93 may be stored in the memory 92. Further, the information stored in the memory 92 may be stored in the storage unit 93.

The operating system is software for mediating between software and hardware, managing a memory space, managing files, and managing processes or tasks, for example. The operating system includes a communication interface. The communication interface is a program for exchanging data with other external devices or the like connected via the communication control unit 96. Examples of the external device or the like include other information processing devices, external storage devices, and the like.

The input unit 94 includes a keyboard, a pointing device, a wireless remote controller, a touch panel, and the like. Further, the input unit 94 can include a video or image input device such as a camera or an audio input device such as a microphone.

The output unit 95 includes a display device such as a liquid crystal display (LCD), an electroluminescence (EL) panel, a cathode ray tube (CRT) display, or a plasma display panel (PDP), or an output device such as a printer. Further, the output unit 95 can include an audio output device such as a speaker.

The communication control unit 96 connects to another device and controls communication between the information processing device 90 and the other device. The communication control unit 96 is, for example, a local area network (LAN) interface board, a wireless communication circuit for wireless communication, and a communication circuit for wired communication. The LAN interface board or the wireless communication circuit is connected to a network such as the Internet.

The information processing device 90 deploys the program stored in the auxiliary storage unit to a work area of a main storage unit using a processor so that the program can be executed, and controls peripheral devices and the like through the execution of the program. Accordingly, the information processing device can realize a function satisfying a predetermined purpose. The main storage unit and the auxiliary storage unit are recording media that can be read by the information processing device.

### Operation Example

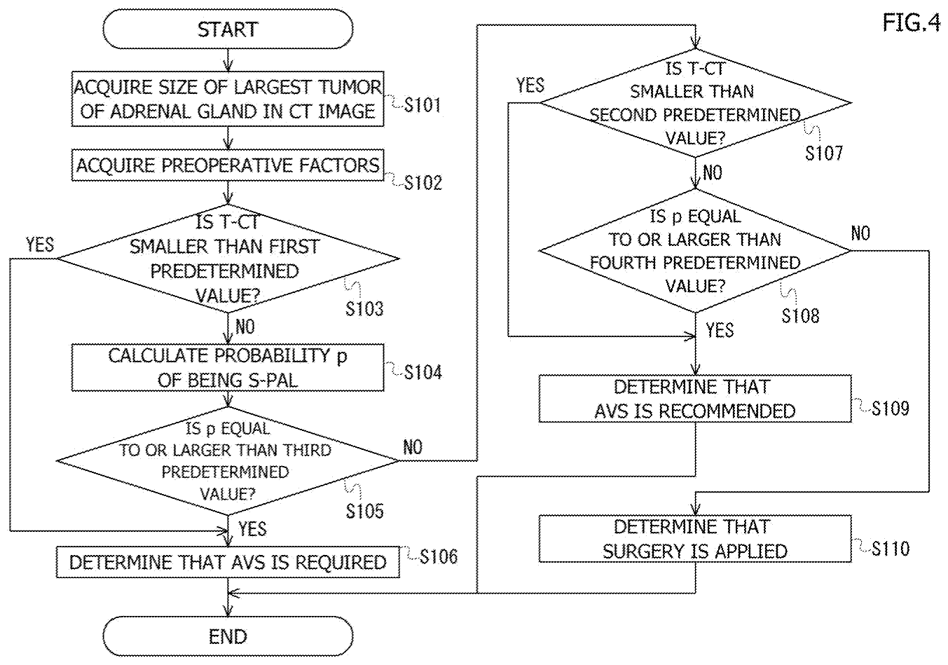

FIG. 4 is a diagram illustrating an example of an operation flow of calculation of the probability p of the tumor of the adrenal gland of the subject being S-PAL in the determination device. Here, the determination device 100 calculates the probability p of the tumor of the adrenal gland being S-PAL for a patient (subject) with primary aldosteronism (PA). The determination device 100 determines, for example, whether or not AVS is required for the subject on the basis of the probability p or the like. Further, it is assumed that the CT image of the adrenal gland of the subject has been taken by the computed tomography device in advance, and a blood of the subject has been tested by the blood testing device. Here, the determination device 100 performs a determination on the subject who is a patient with PA. It is assumed that whether or not the subject is a PA patient is determined using a well-known diagnostic method or the like.

In S101, the tumor diameter acquisition unit 102 of the determination device 100 acquires the size (tumor diameter) of the tumor of the adrenal gland in the CT image of the adrenal gland of the subject taken by a computed tomography device. The tumor diameter acquisition unit 102 acquires the size (tumor diameter) of the largest tumor among the adrenal tumors on the CT image as the tumor diameter (tumor in CT; T-CT) of the adrenal gland on the CT image. The tumor diameter acquisition unit 102 acquires the tumor diameter of the adrenal gland of the subject in the CT image from the computed tomography device or the like connected to the determination device 100. In this case, it is assumed that the tumor diameter of the adrenal gland of the subject in the CT image is calculated in the computed tomography device or the like. Further, the tumor diameter acquisition unit 102 may acquire the CT image in which a part including the adrenal gland of the subject is imaged, and calculate the tumor diameter of the largest tumor among the adrenal tumors in the CT image through known image processing to acquire the tumor diameter of the adrenal gland in the CT image. Further, the tumor diameter acquisition unit 102 may acquire the tumor diameter by causing the user (doctor or the like) to input the tumor diameter of the adrenal gland in the CT image of the subject using an input means or the like. The acquired tumor diameter is stored in the archive unit 110.

In S102, the preoperative factor acquisition unit 104 of the determination device 100 acquires the independent preoperative factors for predicting S-PAL. Here, it is assumed that the preoperative factors are the serum potassium value of the subject and the plasma aldosterone concentration of the subject. The preoperative factor is not limited to the two types, and may be another factor. It is assumed that the preoperative factor is significantly relevant to whether or not the tumor of the adrenal gland is S-PAL on the basis of statistical analysis or the like. The preoperative factor may be a factor other than factors obtained from blood. The preoperative factor acquisition unit 104 acquires the serum potassium value of the subject and the plasma aldosterone concentration of the subject from the blood testing device or the like connected to the determination device 100. In the blood testing device, the serum potassium value and the plasma aldosterone concentration are detected in blood collected from the subject, which is a preoperative factor, in advance. Further, the preoperative factor acquisition unit 104 may acquire the preoperative factors by causing a user (a doctor or the like) to input the serum potassium value of the subject and the plasma aldosterone concentration of the subject using, for example, an input means such as a keyboard or a touch panel. The acquired preoperative factors are stored in the archive unit 110.

In S103, the calculation unit 106 of the determination device 100 determines whether or not the tumor diameter (T-CT) of the adrenal gland in the CT image is smaller than a first predetermined value. The calculation unit 106 extracts the T-CT and the first predetermined value stored in the archive unit 110, and performs the determination. Here, the first predetermined value is 5 mm. The first predetermined value is a threshold value for determining whether or not the AVS is necessary. When the tumor diameter of the adrenal gland is smaller, it is more difficult to determine whether the tumor is the unilateral APA or the bilateral IHA. Therefore, when T-CT is smaller than the first predetermined value, a determination is made that it is necessary for AVS to be performed. When T-CT is smaller than the first predetermined value (S103; YES), the processing proceeds to S106. When T-CT is equal to or larger than the first predetermined value (S103; NO), the processing proceeds to S104.

In S104, the calculation unit 106 calculates the probability p of the tumor of the adrenal gland of the subject being S-PAL. A calculation formula for calculating the probability p is, for example, the above regression formula. The calculation unit 106 uses the serum potassium value of the subject and the plasma aldosterone concentration of the subject that are the preoperative factors acquired in S102, which are stored in the archive unit 110, to calculate the probability p of the tumor of the adrenal gland of the subject being S-PAL using the calculation formula. The calculation unit 106 stores the calculated probability p in the archive unit 110.

In S105, the calculation unit 106 determines whether or not the probability p calculated in S104 is equal to or larger than the third predetermined value. The calculation unit 106 extracts the probability p and the third predetermined value stored in the archive unit 110, and performs the determination. Here, it is assumed that the third predetermined value is 31.8%. The third predetermined value is determined on the basis of the fact that a percentage of patients of which the probability p is equal to or larger than the third predetermined value is 25% in past cases. When the probability p is equal to or larger than the third predetermined value (S105; YES), the processing proceeds to S106. When the probability p is smaller than the third predetermined value (S105; NO), the processing proceeds to S107. The third predetermined value may be determined on the basis of, for example, whether or not there is a possibility of being bilateral IHA when the probability p is equal to or larger than the third predetermined value on the basis of past cases, statistical analysis, or the like. When there is a possibility of being bilateral IHA, it is preferable for AVS to be performed.

In S106, when the tumor diameter (T-CT) of the adrenal gland in the CT image is smaller than the first predetermined value in the PA patient, it is different for the calculation unit 106 to determine whether the tumor of the adrenal gland is unilateral APA or bilateral IHA, and thus, the calculation unit 106 determines that it is necessary for AVS, which is another test, to be performed. Further, when the probability p is equal to or larger than the third predetermined value, it is difficult for the calculation unit 106 to determine whether the tumor of the adrenal gland is unilateral APA or bilateral IHA, and thus, the calculation unit 106 determines that it is necessary for AVS to be performed. The output unit 108 outputs a determination result by displaying the determination result on a display or the like.

In S107, the calculation unit 106 of the determination device 100 determines whether or not the tumor diameter (T-CT) of the adrenal gland in the CT image is smaller than a second predetermined value. The calculation unit 106 extracts the T-CT and the second predetermined value stored in the archive unit 110, and performs the determination. The second predetermined value is a value equal to or larger than the first predetermined value. Here, the second predetermined value is 9 mm. The second predetermined value is one of threshold values for determining application of surgery. The second predetermined value is determined on the basis of the fact that a percentage of patients of which T-CT is equal to or larger than the second predetermined value is 75% in past cases. When the tumor diameter of the adrenal gland is larger, a possibility of being unilateral APA is higher. When T-CT is smaller than the second predetermined value (S107; YES), the processing proceeds to S109. When T-CT is equal to or larger than the second predetermined value (S107; NO), the processing proceeds to S108. The second predetermined value is an example of the first threshold value.

In S108, the calculation unit 106 determines whether or not the probability p calculated in S104 is equal to or larger than the fourth predetermined value. The fourth predetermined value is a value equal to or smaller than the third predetermined value. Here, the fourth predetermined value is 15.5%. The fourth predetermined value is determined on the basis of the fact that a percentage of patients with the probability p equal to or higher than the fourth predetermined value is 50% in past cases. The calculation unit 106 extracts the probability p and the fourth predetermined value stored in the archive unit 110, and performs the determination. When the probability p is equal to or larger than the fourth predetermined value (S108; YES), the processing proceeds to S109. When the probability p is smaller than the third predetermined value (S108; NO), the processing proceeds to S110. The fourth predetermined value is an example of the second threshold value.

The second predetermined value and the fourth predetermined value, for example, may be determined on the basis of whether or not a probability of being unilateral APA is high in a case in which the probability p is smaller than the fourth predetermined value at T-CT equal to or larger than the second predetermined value on the basis of past cases, statistical analysis, and the like.

In S109, when the tumor diameter (T-CT) of the adrenal gland in the CT image is equal to or larger than the first predetermined value and smaller than the second predetermined value and the probability p is smaller than the third predetermined value in the PA patient, the calculation unit 106 determines that it is recommended to perform AVS, which is another test. Further, even when T-CT is equal to or larger than the first predetermined value and the probability p is equal to or larger than the fourth predetermined value and smaller than the third predetermined value, the calculation unit 106 determines that it is recommended to perform AVS, which is another test. In these cases, because the PA patient is highly likely to have unilateral APA, but is also likely to have bilateral IHA, the calculation unit 106 determines that it is recommended to perform AVS. The output unit 108 outputs a determination result by displaying the determination result on a display or the like.

In S110, the calculation unit 106 determines that surgery is applied when the tumor diameter (T-CT) of the adrenal gland in the CT image is equal to or larger than the second predetermined value and the probability p is smaller than the fourth predetermined value in the PA patient. That is, in this case, the calculation unit 106 determines that it is recommended to surgically remove the adrenal gland with a tumor without performing AVS because the PA patient is likely to have unilateral APA. The output unit 108 outputs a determination result by displaying the determination result on a display or the like.

The determination device 100 may execute the operation flow in FIG. 4 in a changed order of operations as much as possible. Further, for example, when the determination device 100 determines that adrenalectomy is indicated, the determination device 100 may execute only S101, S102, S107, S108, and S110.

(Actual Case)

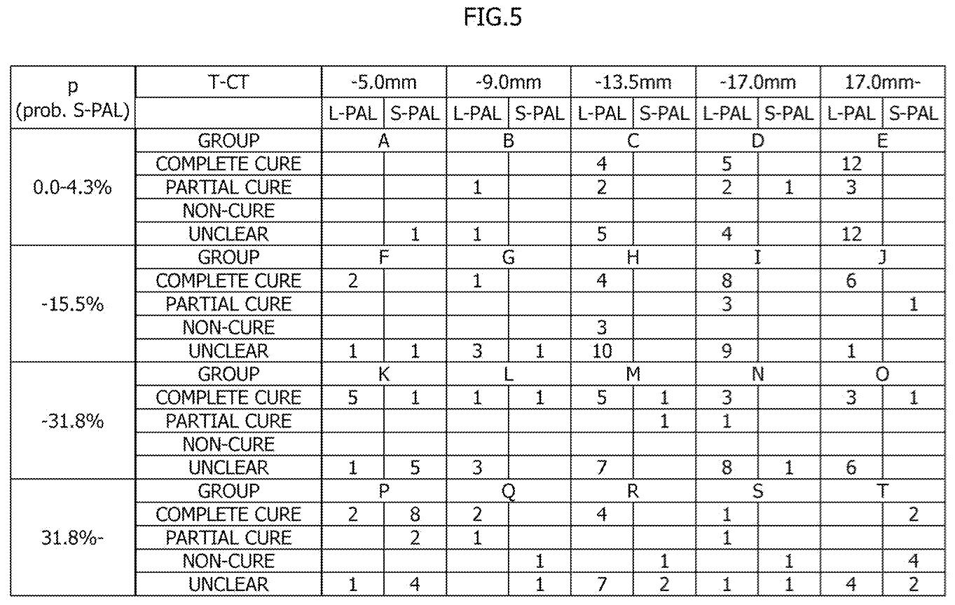

FIG. 5 is a diagram illustrating an actual case. In a table of FIG. 5, cure results of the PA patient subjected to adrenalectomy are classified into T-CT, the probability p of the adrenal tumor being S-PAL, and whether the PA-lesion after adrenalectomy is S-PAL or L-PAL. Treatment results are classified into complete cure, partial cure, non-cure, and unclear. Conditions for complete cure, partial cure, and non-cure are the same as above. T-CT is classified into less than 5.0 mm, 5.0 mm or more and less than 9.0 mm, 9.0 mm or more and less than 13.5 mm, 13.5 mm or more and less than 17.0 mm, and 17.0 mm or more. In examples of disease shown here, a percentage of patients with a T-CT less than 9.0 mm is 25%, a percentage of patients with a T-CT less than 13.5 mm is 50%, and a percentage of patients with a T-CT of less than 17.0 mm is 75%. The probability p is classified into less than 4.3%, 4.3% or more and less than 15.5%, 15.5% or more and less than 31.8%, and 31.8% or more. In the examples of disease shown here, a percentage of patients with the probability p less than 4.3% is 25%, a percentage of patients with the probability of less than 15.5% is 50%, and a percentage of patients with a probability less than 31.8% is 75%.

In the table of FIG. 5, in the case of patients having a T-CT equal to or larger than 13.5 mm (the second predetermined value) and the probability p smaller than 15.5% (the fourth predetermined value) (groups C, D, E, H, I, and J), a result of cure is complete or partial cure in most patients (including patients with S-PAL). Therefore, in this case, it is shown that AVS may be omitted and the adrenalectomy may be performed.

(Action and Effects of Embodiment)

The determination device 100 of the present embodiment acquires preoperative factors of a subject who is a PA patient, and calculates the probability p of the tumor of the adrenal gland of the subject being S-PAL on the basis of the preoperative factors. Examples of the independent preoperative factors for predicting S-PAL include the serum potassium value of the subject and the plasma aldosterone concentration of the subject. The determination device 100 determines, for example, whether or not the AVS is required, whether or not adrenalectomy is indicated on the basis of the probability p and the tumor diameter (T-CT) of the subject in the CT image. The determination device 100 determines whether the adrenalectomy is indicated for a patient with a low probability p of being S-PAL (smaller than the fourth predetermined value) and a large tumor size (T-CT) in the CT images (equal to or larger than the second predetermined value). According to the determination device 100, it is possible to determine that the possibility of being the unilateral APA (adrenalectomy indication) is high for a patient with a low probability p of the adrenal tumor being S-PAL (high possibility of L-PAL) and a large tumor diameter of the adrenal gland in the CT image. AVS may not be performed on the patient. Accordingly, it is possible to reduce the number of PA patients that are subjected to AVS, which is a heavy burden, when a doctor or the like determines whether the adrenalectomy is indicated for the PA patient.

<Computer-Readable Recording Medium>

A program that causes a computer or other machine or device (hereinafter referred to as a computer or the like) to realize any one of the above functions can be recorded on a recording medium that can be read by the computer or the like. The function can be provided by causing the computer or the like to read and execute the program in this recording medium.

Here, the recording medium that can be read by the computer or the like is a recording medium that can store information such as data or programs through an electrical, magnetic, optical, mechanical, or chemical action and can be read from the computer or the like. In such a recording medium, elements constituting a computer, such as a CPU and a memory, may be provided, and the CPU may be caused to execute the program.

Among such recording media, examples of the recording media that can be removed from the computer or the like include a flexible disc, a magneto-optical disc, a CD-ROM, a CD-R/W, a DVD, a DAT, an 8 mm tape, and a memory card.

Further, there are a hard disk, a ROM, and the like as recording media fixed to the computer or the like.

(Others)

Although the embodiments of the present invention have been described above, these are merely examples, and the present invention is not limited thereto, and various changes based on knowledge of those skilled in the art such as a combination of respective configurations can be made without departing from the gist of the claims.

### REFERENCE SIGNS LIST

100 Determination device
102 Tumor diameter acquisition unit
104 Preoperative factor acquisition unit
106 Calculation unit
108 Output unit
110 Archive unit
90 Information processing device
91 Processor
92 Memory
93 Storage unit
94 Input unit
95 Output unit
96 Communication control unit

1. A determination device comprising:
a factor information acquisition unit configured to acquire information on one or more predetermined factors for a subject with primary aldosteronism;
a tumor diameter acquisition unit configured to acquire a tumor diameter, the tumor diameter being a size of a largest tumor among tumors included in an image of an adrenal gland of the subject obtained through computed tomography; and
a calculation unit configured to calculate a probability of an aldosterone-producing adenoma of the adrenal gland of the subject being smaller than a predetermined size on the basis of the information of the one or more predetermined factors acquired by the factor information acquisition unit, and determine that adrenalectomy is indicated for the subject on the basis of the tumor diameter and the probability.

2. The determination device according to claim 1, wherein the one or more predetermined factors include a serum potassium value and a plasma aldosterone concentration.

3. The determination device according to claim 1, wherein the calculation unit determines that adrenalectomy is indicated for the subject when the tumor diameter is equal to or larger than a first threshold value and the probability is smaller than a second threshold value.

4. A determination method executed by a computer, the method comprising:
acquiring information on one or more predetermined factors for a subject with primary aldosteronism;
acquiring a tumor diameter, the tumor diameter being a size of a largest tumor among tumors included in an image of an adrenal gland of the subject obtained through computed tomography; and
calculating a probability of an aldosterone-producing adenoma of the adrenal gland of the subject being smaller than a predetermined size on the basis of the information of the one or more predetermined factors acquired by the factor information acquisition unit, and determining that adrenalectomy is indicated for the subject on the basis of the tumor diameter and the probability.

5. A non-transitory computer readable medium storing a determination program for causing a computer to execute:
acquiring information on one or more predetermined factors for a subject with primary aldosteronism;
acquiring a tumor diameter, the tumor diameter being a size of a largest tumor among tumors included in an image of an adrenal gland of the subject obtained through computed tomography; and
calculating a probability of an aldosterone-producing adenoma of the adrenal gland of the subject being smaller than a predetermined size on the basis of the information of the one or more predetermined factors acquired by the factor information acquisition unit, and determining that adrenalectomy is indicated for the subject on the basis of the tumor diameter and the probability.
